(19) United States
(12) Patent Application Publication
(10) Pub. No.: US 2013/0243151 A1
(43) Pub. Date: Sep. 19, 2013

(54) PORTABLE X-RAY COMPUTED TOMOGRAPHY (CT) SCANNER

(71) Applicant: Ang Shih, San Diego, CA (US)

(72) Inventor: Ang Shih, San Diego, CA (US)

(73) Assignee: HALO IMAGING, INC., San Diego, CA (US)

(21) Appl. No.: 13/777,788

(22) Filed: Feb. 26, 2013

Related U.S. Application Data
(60) Provisional application No. 61/604,363, filed on Feb. 28, 2012.

(51) Int. Cl. A61B 6/03

(52) U.S. Cl. CPC A61B 6/035
USPC 378/9; 378/4; 29/428; 29/592.1

(57) ABSTRACT

The present invention provides a portable x-ray CT scanner for generating diagnostically meaningful images of a subject, such as a patient's cranium. The system includes a gantry ring which is configured to be portable or hand-held which is tether to a power supply. The device allows for simple, quick scans which are compensated for movement artifact and may then be transmitted electronically to local or regional medical facilities in advance of the patient's arrival.

### CROSS-REFERENCE TO RELATED APPLICATION(S)

This application claims benefit of priority under 35 U.S.C. §119(e) of U.S. Ser. No. 61/604,363, filed Feb. 28, 2012, the entire contents of which are incorporated herein by reference in its entirety.

### BACKGROUND OF THE INVENTION

1. Field of the Invention

The present invention relates generally to medical imaging and more specifically to a mobile x-ray computed tomography (CT) scanning device.

2. Background Information

X-rays penetrating biological tissue are absorbed in proportion to the electron density of the tissue elements. Imaging media ranging from photographic film to solid-state electron-sensing materials placed on the opposite side of the tissue can sense the remaining signal and record the two-dimensional shadows of the tissue elements (FIG. 1). This is known as a plain x-ray (also called plane or planer x-ray) image and is characteristically used to diagnose bone trauma, pleural effusions and extravasated blood. Soft tissue elements such as blood vessels or viscera can be visually enhanced by injection of radio-opaque dyes composed of iodinated compounds or barium prior to imaging.

X-ray imaging is an important medical tool in diagnostic radiography, but it has also been widely used by scientists in crystallography and by industry for inspection of parts and the integrity of mechanical joints. The resolution of x-ray imaging is related to the wavelength of the x-ray, which is in the range of 0.01 to 10 nanometers, between gamma and ultraviolet rays within the electromagnetic spectrum. By comparison, visible light has a wavelength of 1000 nanometers (1 micron), and this limits light microscopy resolution to 1 micron. The depth of x-ray penetration depends directly on the generated energy of the x-ray beam, which typically is within the range of 120 eV to 120 keV.

Medical imaging with x-rays has been greatly enhanced by application of the mathematical process known as tomography, defined as a method of producing a three-dimensional image of the internal structures of a solid object by combining numerous two-dimensional images, each generated and recorded from a stable radial point along a perpendicular (z) axis (FIG. 2). The technique is known as x-ray computed tomography (CT) and is used in systems known as CT scanners or CAT (computer axial tomography) scanners, and has become the standard for diagnostic radiology of the head. This technique effectively eliminates shadow obstructions of tissue elements from separate two-dimensional x-rays. The resulting composite image, or slice if a sequence of axial images is recorded, reveals tissues that differ in physical density by less than 1%.

CT scanning has become an ubiquitous tool in the radiology departments of medical centers, and emergency room requests for their use are frequent. With respect to head injury, the ability to identify intracranial injury and differentiate between epidural, subdural, subarachnoid, parenchymal or ventricular bleeding is vital to the effective management of the patient. CT imaging is typically required to make the diagnosis, but the complexity and physical size of existing CT equipment often limits its availability and can cause delays in defining treatment strategies. Conventional high-resolution CT scanners weigh 6000 pounds or more, require high-voltage power supplies with cooling systems, and are located in climate-controlled radiology suites (FIG. 3). Smaller, so-called portable, CT scanners have been developed for use in the hospital ward or emergency room setting, but these are also massive, and are portable only in the sense that they have wheels. They are not intended or capable of being used in mobile vehicles or moved outside the hospital environment.

The physical size of CT scanners has been driven by the desire to produce the highest resolution images in the least amount of time from any anatomical section of the body. Thus, in the typical configuration, a CT scanning system is composed of a gurney-like table on which the patient lies prone that can be moved through a circulating coil of x-ray generators and imaging devices. The coil rotates once about the long axis of the body to produce one CT image (transverse section). While originally taking up to 10 seconds per revolution, modern slip-ring technology allows the coil gantry mechanism to continuously rotate around the patient at rates up to 30 RPM while the patient table is being advanced. This helical-based data acquisition technique has been further refined by the simultaneous use of dual x-ray scanners operating at different energies and dual detectors, which has enhanced differentiation of mass density.

Accordingly, it would be advantageous to provide a mobile x-ray CT device which retains sufficient image resolution for diagnostic use.

### SUMMARY OF THE INVENTION

In accordance with the present invention, an easily transportable mobile medical imaging system is disclosed. The system is a portable x-ray CT system for providing diagnostically useful images of a patient.

Accordingly, in one aspect, the present invention provides a portable medical imaging system. The system includes a gantry ring having a bore, an image collection apparatus having an x-ray source and an x-ray detector array, a processor comprising computer-executable instructions, and a power source. In various embodiments, the image collection apparatus is rotatably coupled to the gantry ring. In various embodiments, the processor comprises computer-executable instructions for controlling acquisition of a projection of an object produced by the image collection apparatus; controlling movement of the image collection apparatus; and generating an image by integrating multiple projections of the object. In various embodiments, the system further includes one or more sensors coupled to the gantry ring capable of detecting changes in location to account for movement of the gantry during scanning. The sensor may include a camera, accelerometer, global positioning system (GPS), laser, optical sensor, non-ionizing radiation sensor, linear encoder, triangulation sensor, stereo camera, or combination thereof. In various embodiments, the system is compact to allow for portability. As such the gantry ring is less than about 20 inches in diameter and has a width of less than about 10 inches. In various embodiments, the system may further include a collimator operatively coupled to the x-ray source. In one embodiment, the system is hand-held.

In another aspect, the present invention provides a method of imaging an object using an imaging system as described above. The method includes advancing a gantry ring over an object to position the object within the bore; rotating an image collection apparatus rotatably coupled to the gantry ring circumferentially around the object, acquiring projections of the object via the image collection apparatus as the image collection apparatus is rotated around the object; generating an image of the object by analyzing the acquired projections; and transmitting the image. In various embodiments, the object is a human patient, such as a human cranium. In various embodiments, the image is wirelessly transmitted to a remote location, such as to a patient care facility.

### BRIEF DESCRIPTION OF THE DRAWINGS

Features and advantages of the present invention will be apparent from the following detailed description of the invention, taken in conjunction with the accompanying drawings of which:

FIG. 1 is a schematic of a conventional x-ray system using a single x-ray source and detector;

FIG. 2 is a schematic of a conventional x-ray CT system for head imaging using a single x-ray source and detector that is rotated around the patient;

FIG. 3 is a schematic of a conventional commercial CT system;

FIG. 4 is a diagram of a gantry ring in one embodiment of the invention;

FIG. 5 is a block diagram of the system architecture in one embodiment of the present invention;

FIG. 6 is a diagram comparing a detector array and x-ray source configuration of a convention imaging system and system in one embodiment of the invention;

FIG. 7 is a diagram comparing a detector array and x-ray source configuration of a convention imaging system and system in one embodiment of the invention;

FIG. 8 is a diagram including a coordinate map for a system in one embodiment of the invention;

FIG. 9 is a diagram depicting stabilization of vibration for a system in one embodiment of the invention;

FIG. 10 is a diagram depicting active location sensors for use with a system in one embodiment of the invention;

FIG. 11 is a diagram representing algorithm reconstruction for use with a system in one embodiment of the invention; and

FIG. 12 is a diagram representing an interpolation algorithm for use with a system in one embodiment of the invention.

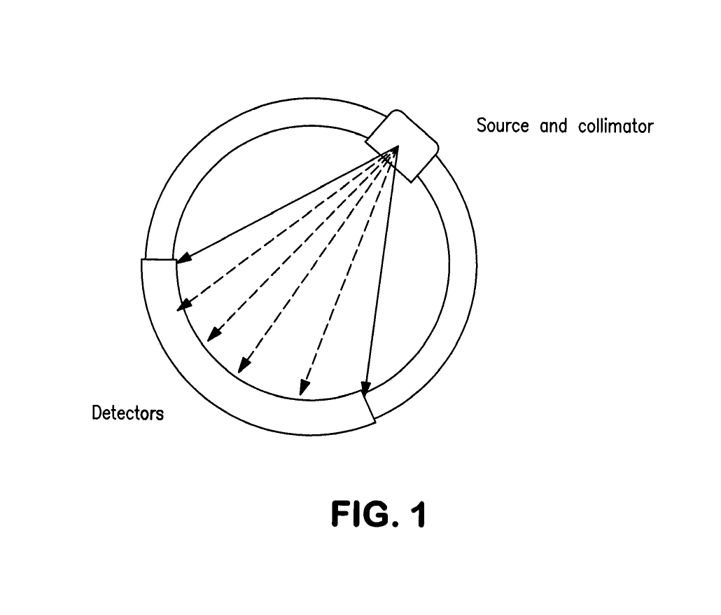

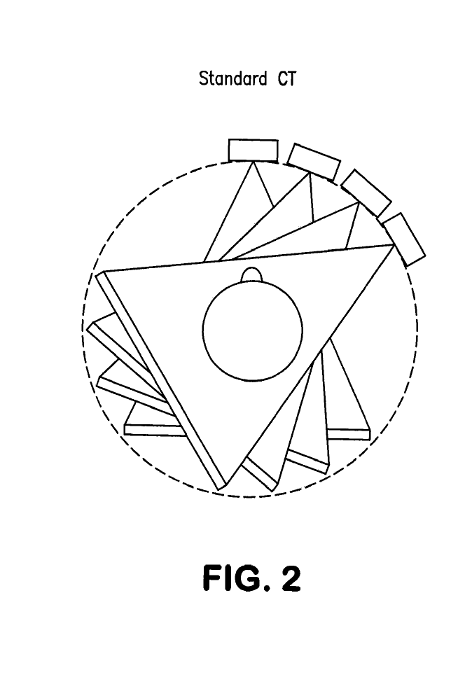

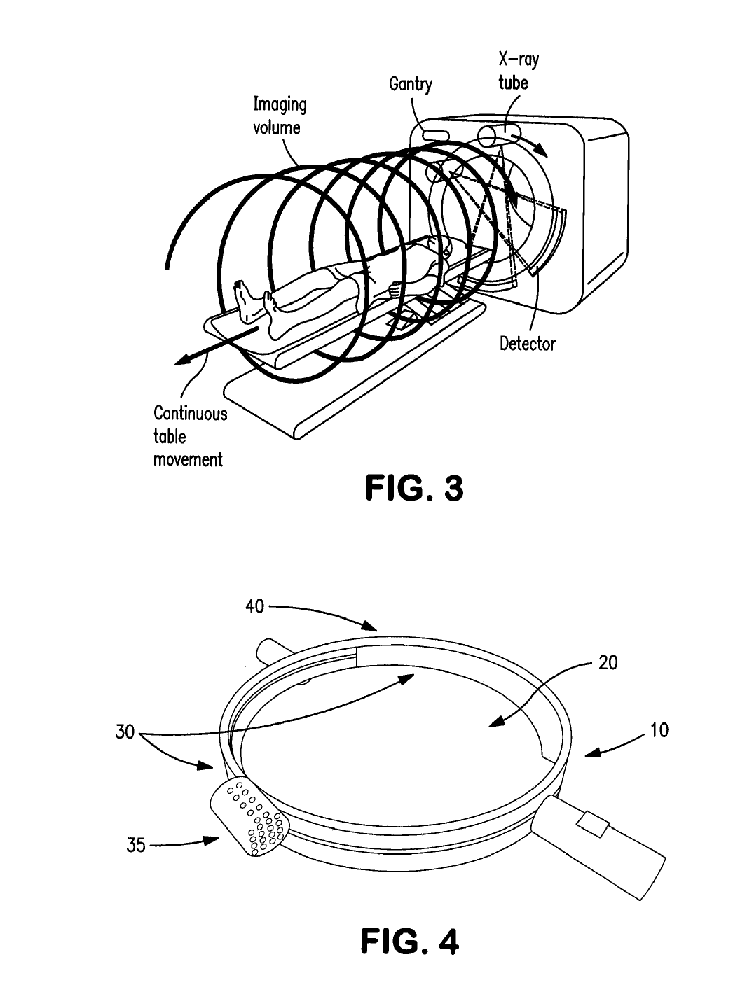

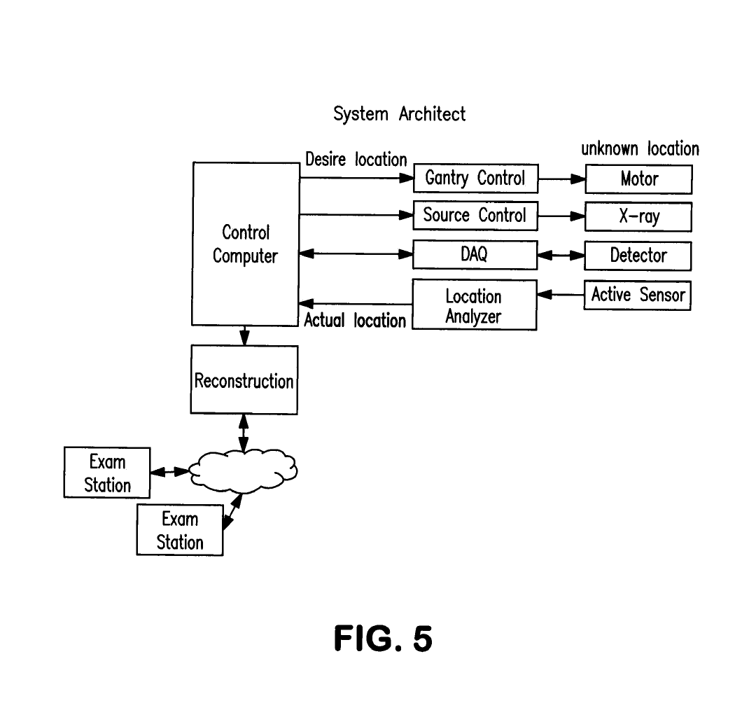

### DETAILED DESCRIPTION OF THE INVENTION

The present invention provides a portable x-ray CT system as well as methods of use thereof. The invention addresses the need for a miniaturized mobile CT system which may be used, for example, in ground/air ambulances or field hospitals for imaging of the skull and cranial contents. The device allows for simple, quick scans which are compensated for movement artifact and transmitted electronically to local or regional medical facilities in advance of the patient's arrival. The information will significantly facilitate the development of a treatment plan for brain injury, which will save critical time once the patient reaches the hospital

Before the present device and method are described, it is to be understood that this invention is not limited to particular device and methodology described. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the device” includes one or more devices of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention provides a portable medical imaging system. With reference to FIG. 4, the system generally includes a gantry ring 10 having a bore 20, an image collection apparatus 30 having an x-ray source 35 and an x-ray detector array 40, a processor comprising computer-executable instructions, and a power source. The x-ray source 35 is a low energy x-ray to which a collimator may be operatively coupled. In various embodiments, the system may be hand-held.

The image collection apparatus 30 is rotatably coupled to the gantry ring 10. This allows for the image collection apparatus to circumferentially travel around an object positioned in the center of the bore 20 during scanning to generate individual projections. The gantry ring 10 is configured as a portable unit to facilitate ease of use by the operator. For example, in embodiments, it may be a hand-held unit.

In use, the gantry ring 10 is advanced over an object, such as the body part of a patient, to position the object within the bore 20. The image collection apparatus 30 which is rotatably coupled to the gantry ring 10, is circumferentially rotated around the object. Projections of the object are acquiring via the image collection apparatus as the image collection apparatus 30 is rotated around the object.

In various embodiments, at least one projection is generated per second to allow scanning of a discrete region of the object in a duration of between 10 to 15 seconds. The individual projections are analyzed via functionality of a computer processor to generate a diagnostically useful image which may then be transmitted to an on-board visual display or to a remote location, such as a patient care facility. In some embodiments, at least one projection is generated each 0.25, 0.5, 0.75, 1.0, 1.25, or 1.5 seconds.

To facilitate operation of the device and acquisition of images, the system includes a processor having computer-executable instructions for performing such tasks. For example, the computer-executable instructions may be for controlling acquisition of a projection of an object produced by the image collection apparatus 30; controlling movement of the image collection apparatus 30; and generating an image by integrating multiple projections of the object. In various embodiments, the system may further includes a visual display to allow the images to be displayed. However, the system does not require a visual display, for example in embodiments where images and/or other data is wirelessly transmitted to a care facility. Additionally, the system may include functionality to wirelessly link to the operator's cellular device, smartphone, or the like to display images.

The device is lightweight and portable to facilitate operation in non-traditional settings such as an ambulance or in the field as opposed to a long term care facility. In various embodiments the device is less than about 20 pounds in weight. In some embodiments, the device is less than or equal to about 20, 19, 18, 17, 16, 15, 14, 13, 12, 11, 10, 9 or 8 pounds.

Furthermore, the physical size of the device is no larger than a backpack including the tethered gantry for portability. In various embodiments, the gantry ring 10 is less than about 20 inches in diameter. For example, the gantry ring 10 may be less than or equal to about 10-20, 10-18, 10-16, 10-15, 10-14, 10-13 or 10-12 inches in diameter. Additionally, in various embodiments, the gantry ring 10 is less than about 10 inches in width. For example, the gantry ring 10 may be less than or equal to about 2-10, 4-10 or 4-8 inches in width.

Generally, the gantry ring 10 is circular in shape. However, the gantry ring 10 may be oval or elliptical in shape to accommodate specifically shaped objects.

The system may be powered by an external power source, such as a vehicle DC or AC electrical system. Additionally, the power source may be a rechargeable battery supply for use in venues remote from a vehicle.

In various embodiments, the system may utilize multiple image collection apparatuses 30 which include multiple x-ray sources 35 as well as multiple x-ray detector arrays 40. For example, in one embodiment, the system utilizes at least two x-ray sources 35 and corresponding x-ray detectors 40. The source and detectors may be placed in an off-center configuration to produce a comprehensive CT scan. Multiple image collection apparatuses 30 effectively reduce the physical size and circumference of the imager, the scan rotation, x-ray production and energy utilization.

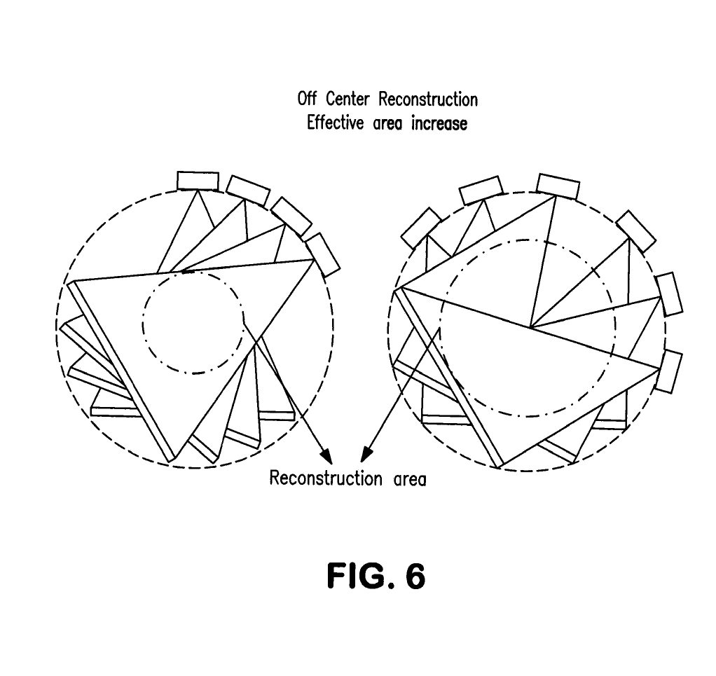

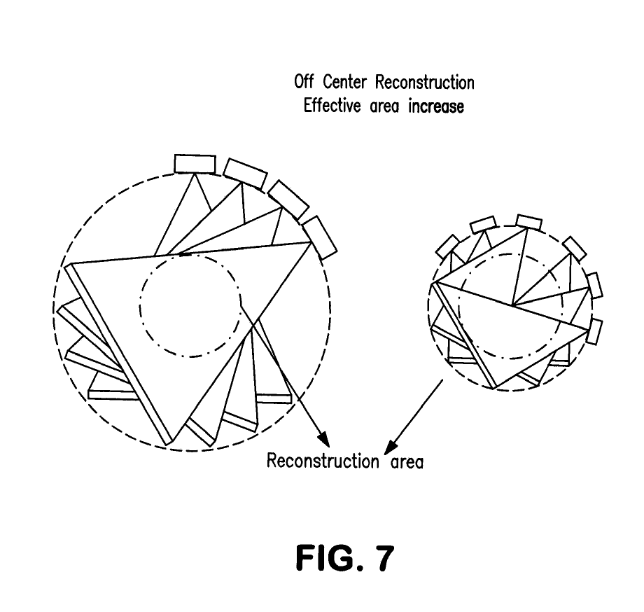

With reference to FIG. 6, utilization of off-center detector(s) and multiple x-ray sources increase scanner area thereby allowing a smaller physical ring size. On the left of FIG. 6, a conventional CT system is shown with one detector and source. Ideally, the source is far enough away from the patient so that the x-ray projection covers the entire desired area of the patient. On the right of FIG. 6, the system of the present invention includes at least two sources with offset angles pointing at one detector array so that the combination of projections from both sources covers a larger effective area of the patient. This allows both sources to be closer to the patient, thereby reducing the ring size requirement as compared to a conventional CT system. FIG. 7 shows that a reduced ring size of the system of the present invention shown on the right is made possible by offset x-ray sources.

Accordingly, in some embodiments, an image collection apparatus for use in the present invention may include a plurality of x-ray sources for each x-ray detector. For instance, 2, 3, 4, 5, 6, 7, 8, 9 or 10 x-ray sources may be provided for a single x-ray detector.

The angle of offset between projections from x-ray sources may be between about plus or minus 30° from the normal line at the point on the gantry ring where the x-ray source is located. In various embodiments, the angle of offset may be less than or equal to plus or minus 30°, 25°, 20°, 15°, 10° or 5° from the normal line at the point on the gantry ring where the x-ray source is located.

The system of the present invention is further capable of active stabilization by accounting for relative changes in location of the gantry ring 10 relative to the object being imaged such that the system may be utilized in any environment in which vibrations occur. In part, this is achieved by including one or more sensors coupled to the gantry ring 10 capable of detecting changes in location of the gantry ring 10 relative to the object.

In embodiments, active stabilization allows the system to be adapted for use in mobile environments, such as in an automobile, aircraft, helicopter or watercraft. However, it will be appreciated that active stabilization may be utilized in non-mobile environments as well. For example, active stabilization may be included to address any movement artifact (caused by, for instance, operator handheld usage) even in a non-mobile environment.

One skilled in the art would understand that any number and type of sensor may be utilized with the system which allow for spatial determinations by, for example, direct viewing, triangulation or the like. Such sensors may be operatively positioned or coupled to the gantry ring 10. By way of illustration, such sensors include wherein the sensor comprises a camera, accelerometer, global positioning system (GPS), laser, optical sensor, non-ionizing radiation sensor, linear encoder, triangulation sensor, stereo camera, or combination thereof.

In one embodiment, the system may include one or more linear encoders, for example at the supporting joint(s) of the lever. Such encoders may be mechanical, capacitance, inductance, optical based encoders or the like. In some embodiments, the sensor may be capable of approximate sensing utilizing, for example, capacitance, inductance, Hall effect, or Doppler effect. In some embodiments, the sensor allows for triangulation via, for example timestamps, GPS, non-ionizing radiation, such as RF, ultrasonic, visible light, IR, UV, and sound waves. In one embodiment, the sound source may be mounted on gantry ring 10, or reference location, or both. In some embodiments, the sensor may utilize cameras, such as stereo cameras or cameras utilizing tracking algorithms. In some embodiments, the sensor may employ a “light curtains technique” using any number of wavelengths or physical properties, such as capacitance, inductance, optics, sound waves and non-ionizing radiation.

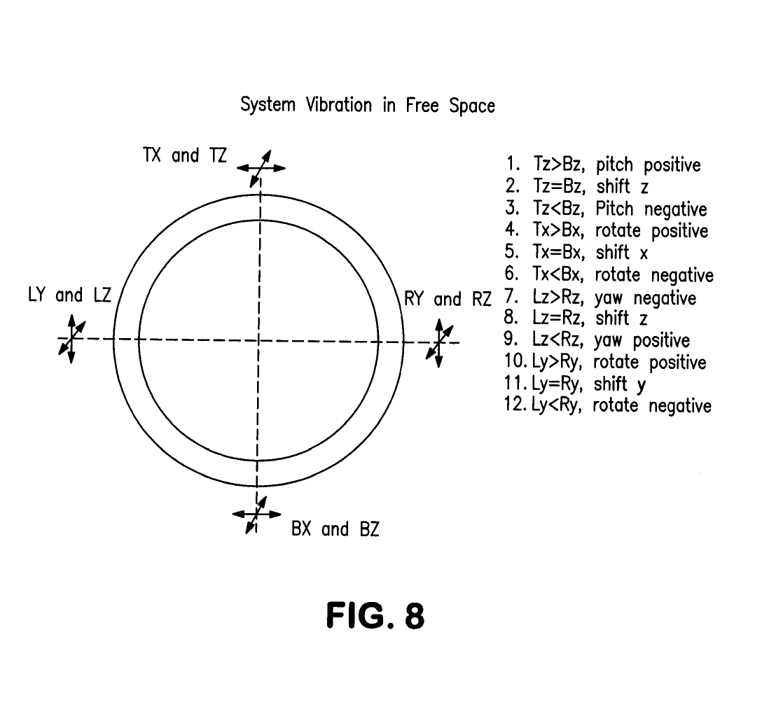

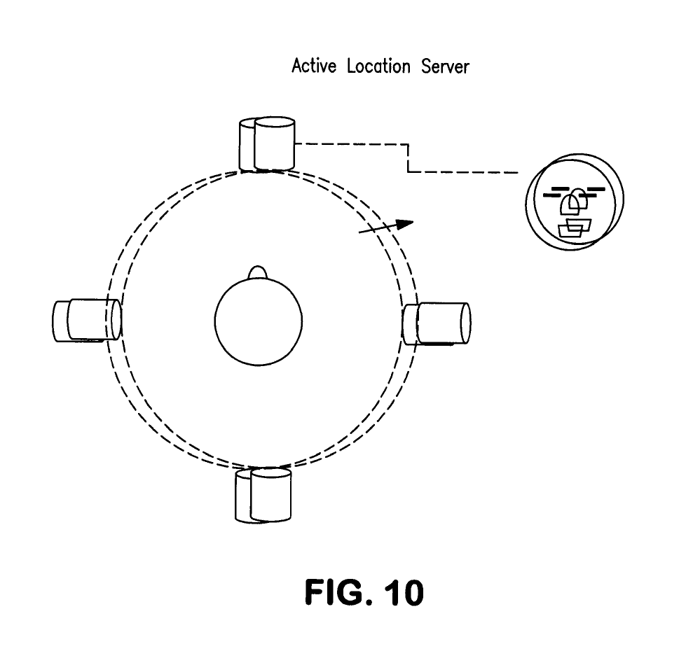

In various embodiments, the relative motion of the object, e.g., the patient, with respect to the x-ray detector array 40 is tracked with one or more small cameras and accelerometers operatively coupled to the gantry ring 10, allowing scans to be performed and corrected for movement artifact. (FIGS. 7-10).

Movement of the CT system or the patient is an issue that has prevented existing CT systems from being used in a capacity in which movement artifacts are present, for example in a mobile or hand-held capacity. Active location sensory are used with the present invention to detect the type of movement and relative position of the system to the patient. Then, the positions are used to calculate the appropriate reconstruction of the stable projection. In various embodiments, 1, 2, 3, 4, 5, 6, 7, 8, 9, 10 or more active location sensors may be utilized. During reconstruction, the x-ray sources 35 and x-ray detectors 40 may be at different locations during each projection.

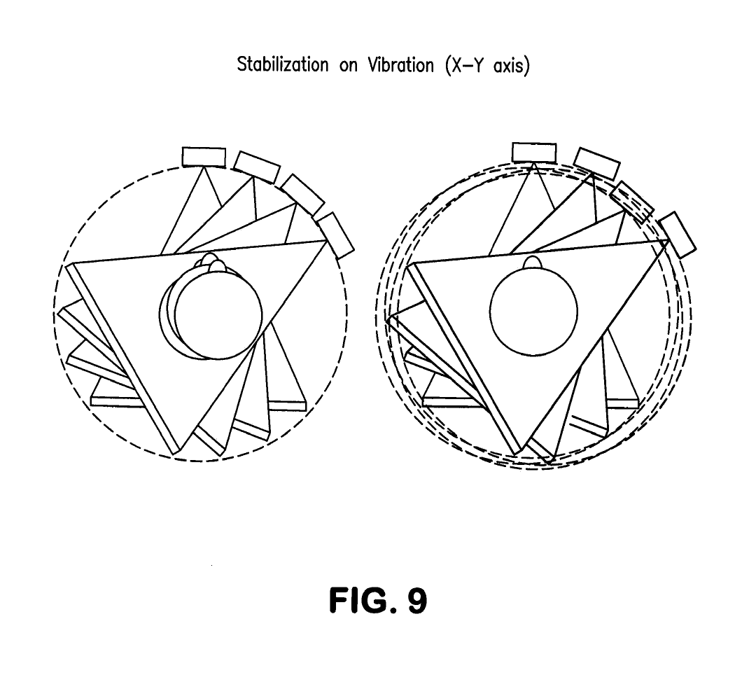

Active location sensors monitor the relative change in position between the system and patient in both 2 and 3 dimensions. In addition, the type of movement occurring from the patient or the system is obtained by the active location sensors. Relative position of the patient to the system is captured. Therefore, the x-ray source 35 and x-ray detector 40, or the patient may be at different locations during each x-ray projection. The positions of the source 35, detector 40, and patient are then used to calculate the reconstruction of the image. The active location sensors detect all movement that is used for 2D compensation (X axis, Y axis, rotation against Z axis) or 3D compensation (Z axis, rotation against X axis and rotation against Y axis). FIG. 9 depicts stabilization in two dimensions.

In various embodiments, the system may further include one or more cameras. Camera may be mounted on the gantry separate from the image collection apparatus 30, or alternatively they may be integral with the image collection apparatus.

For example, in one embodiment, a camera is mounted to the gantry ring 10 of the system. In one embodiment, the camera is a digital camera, or in another embodiment, the camera is a web camera. However, as will be appreciated by those in the art, any type of camera can be employed.

In embodiments, photographic images of an object are acquired as the camera rotates about the object with the image collection apparatus 30. The external images acquired by the camera may each correlate to one of the x-ray images taken by the image collection apparatus 30. For instance, the camera captures an external image of the object at each of a plurality of rotational positions. This allows the camera to record a real time image of the object marking where the x-rays from the x-ray source are being directed.

Alternately, each of the external images may not correlate exactly to one of the x-ray images. However, the x-ray images and the external images are taken at known relative positions as determined by accompanying sensors. The camera takes numerous external images of the object as it rotates upon the gantry ring 10, and the computer processor associates each of the external images to one of the x-ray images based on known relative positions determined by positional sensors.

The external images are provided to the computer processor. A three dimensional external image may be generated from the plurality of external images. The three dimensional external image is registered relative to the three dimensional x-ray derived image generated from the plurality of x-ray images. For instance, the three dimensional external image and the three dimensional CT image are overlapped.

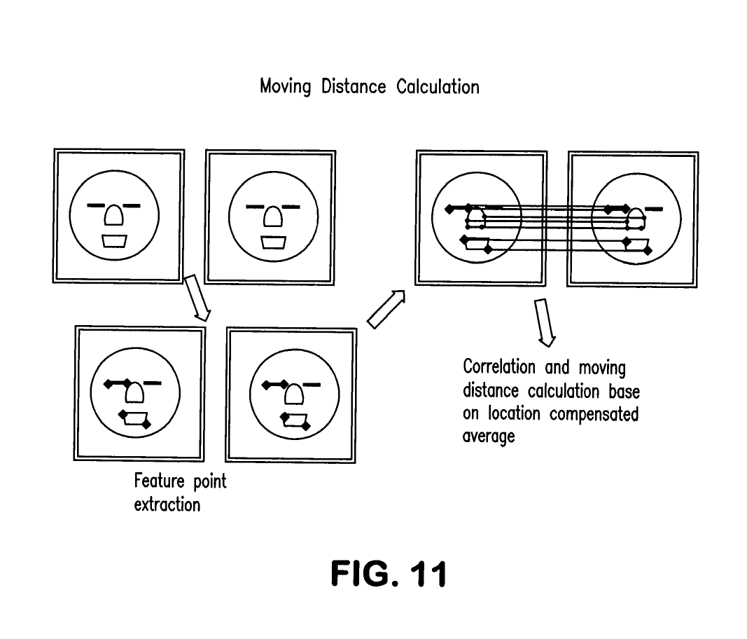

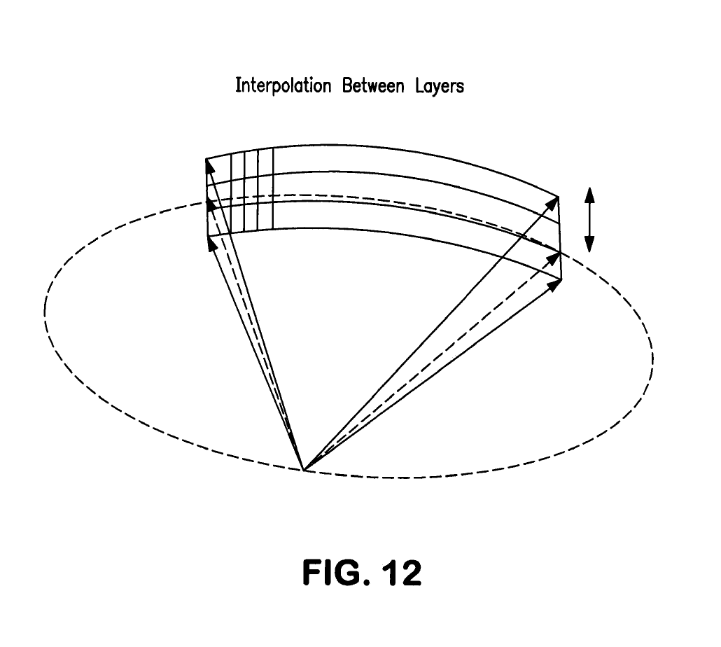

The processor of the present invention is provided with functionality to integrate and analyze data obtained from active stabilization sensors and x-ray projections to produce a robust image of the object. The processor is provided with functionality to perform algorithms to integrate scan geometry and active stabilization, as well as optimize the image to emphasize bone, metal and blood in brain. With reference to FIG. 11, in one embodiment an algorithm reconstruction in 2D is used to compensate for movement in 2D (X axis, Y axis, rotation against Z axis). Use of future point extraction may then be utilized to correlate distance between existing points and future points. With reference to FIG. 12, interpolation may be utilized to cancel out movement in 3D (Z axis, rotation against X axis and rotation against Y axis).

In various embodiments, a typical reconstruction algorithm that may be utilized to reconstruct projections from different locations and movements in 2D and 3D is as follows:

$$f ( x , y , {z}_{t} ) = \text{?} \frac{1}{L 2} \text{?} \hat{P} ( β , θ , v ) g ( \tilde{θ} - θ ) \frac{{R}_{f}}{\sqrt{{R}_{f}^{2} + {v}^{2}}} d θ d β , \\ \text{?} \text{indicates text missing or illegible when filed}$$

where ƒ(x, y, zₜ) is a function to be reconstructed at the point A: (x, y, zₜ)^{T},

L=\sqrt{(x−xₛ)²+(y−yₛ)²}.

\hat{P}(β, θ, ν)=ω(α, β, θ)P(β, θ, ν)R_{ƒ} cos θ,

Additional algorithms are well known in the art and suitable for image restoration. Such algorithms include tilt reconstruction algorithms as well as those discussed on the world wide web at URL ‘imaging.sbes.vt.edu/teaching/ct-image-restoration/’ which is incorporated herein in its entirety.

Images that are generated by the system are transmitted to a local visual display or remote monitoring station. As such, the system may further include one or more data communication links. Each such data communication link may include a wired or wireless communication path which may be configured for secure, encrypted uni- or bi-directional data exchange. In particular, the data communication link in one embodiment may include radio data communication, satellite data communication, Wi-Fi data communication, IrDA data communication, infrared data communication, Bluetooth™ data communication, ZigBee™ data communication, USB or Firewire cable based data communication, Ethernet cable based data communication, and dial up modem data communication.

It will be understood that the system described herein may be constructed of any sufficiently rigid and strong materials such as high-strength plastic, metal, carbon fiber and the like, as well as combinations of the same.

As used herein, the term “patient” or “subject” refers to a variety of animal types. Generally the patient or subject is human, although as will be appreciated by those in the art, the patient or subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition.

Although the invention has been described with reference to the above example, it will be understood that modifications and variations are encompassed within the spirit and scope of the invention. Accordingly, the invention is limited only by the following claims.

What is claimed is:

1. A portable medical imaging system, comprising:
(a) a gantry ring having a bore and an image collection apparatus, the image collection apparatus comprising an x-ray source and an x-ray detector array, wherein the image collection apparatus is rotatably coupled to the gantry ring, and wherein the gantry ring is less than about 20 inches in diameter and less than about 10 inches in width; and
(b) a processor comprising computer-executable instructions for:
(i) controlling acquisition of a projection of an object produced by the image collection apparatus;
(ii) controlling movement of the image collection apparatus; and
(iii) generating an image by integrating multiple projections of the object; and
(c) a power source.

2. The medical imaging system of claim 1, further comprising one or more sensors coupled to the gantry ring capable of detecting changes in location.

3. The medical imaging system of claim 2, wherein the sensor comprises a camera, accelerometer, global positioning system (GPS), laser, optical sensor, non-ionizing radiation sensor, linear encoder, triangulation sensor, stereo camera, or combination thereof.

4. The medical imaging system of claim 1, wherein the image collection apparatus rotates 360 degrees around the interior of the gantry ring to obtain projections of an object positioned within the bore.

5. The medical imaging system of claim 1, wherein the gantry ring is about 10 to 15 inches in diameter.

6. The medical imaging system of claim 1, wherein the gantry ring has a width of about 4 to 8 inches.

7. The medical imaging system of claim 1, further comprising a data transmission portal for wireless transmission of data.

8. The medical imaging system of claim 1, wherein at least one projection is acquired each second.

9. The medical imaging system of claim 1, wherein the gantry ring is tethered to the power supply via a flexible wire.

10. The medical imaging system of claim 1, wherein the system is less than about 20 pounds in weight.

11. The medical imaging system of claim 1, wherein generation of the image is accomplished via a reconstruction algorithm, interpolation, or a combination thereof.

12. The medical imaging system of claim 1, wherein the x-ray detector array comprises at least 2 detectors.

13. The medical imaging system of claim 1, wherein the image collection apparatus comprises multiple x-ray sources.

14. The medical imaging system of claim 13, wherein the image collection apparatus comprises multiple x-ray detector arrays.

15. The medical imaging system of claim 1, wherein the object is a human patient.

16. The medical imaging system of claim 1, wherein the object is a human cranium.

17. The medical imaging system of claim 1, further comprising a collimator operatively coupled to the x-ray source.

18. A method of imaging an object using a portable medical imaging system, comprising:
(a) advancing a gantry ring over an object to position the object within the bore;
(b) rotating an image collection apparatus rotatably coupled to the gantry ring circumferentially around the object, wherein the image collection apparatus comprises an x-ray source and an x-ray detector array, and wherein the gantry ring is adapted to be hand-held by a user and is less than about 20 inches in diameter and less than about 10 inches in width;
(c) acquiring projections of the object via the image collection apparatus as the image collection apparatus is rotated around the object;
(c) generating an image of the object by analyzing the projections acquired in (c); and
(d) transmitting the image.

19. The method of claim 18, wherein the object is a human patient.

20. The method of claim 18, wherein the object is a cranium of a human patient.

21. The method of claim 18, wherein the device comprises a processor having computer-executable instructions for:
(i) controlling acquisition of the projection of the object produced by the image collection apparatus;
(ii) controlling movement of the image collection apparatus; and
(iii) generating the image by integrating multiple projections of the object.

22. The method of claim 18, wherein the image is wirelessly transmitted.

23. The method of claim 18, wherein the image is transmitted to a patient care facility.

24. The method of claim 18, wherein the generation of the image is accomplished via a reconstruction algorithm, interpolation, or a combination thereof.

25. The method of claim 18, wherein the imaging system, further comprises one or more sensors coupled to the gantry ring capable of detecting changes in location.

26. The method of claim 25, wherein the sensor comprises a camera, accelerometer, global positioning system (GPS), laser, optical sensor, non-ionizing radiation sensor, linear encoder, triangulation sensor, stereo camera, or combination thereof.

27. The method of claim 18, wherein the image collection apparatus rotates 360 degrees around the interior of the gantry ring to obtain projections of the object positioned within the bore.

28. The method of claim 18, wherein the gantry ring is about 10 to 15 inches in diameter.

29. The method of claim 18, wherein the gantry ring has a width of about 4 to 8 inches.

30. The method of claim 18, wherein at least one projection is acquired each second.

31. The method of claim 18, wherein the gantry ring is tethered to a rechargeable power supply via a flexible wire.

32. The method of claim 18, wherein the imaging system is less than about 20 pounds in weight.

33. The method of claim 18, wherein the x-ray detector array comprises at least 2 detectors.

34. The method of claim 18, wherein the image collection apparatus comprises multiple x-ray sources.

35. The method of claim 34, wherein the image collection apparatus comprises multiple x-ray detector arrays.

36. The method of claim 18, further comprising a collimator operatively coupled to the x-ray source.

37. A method of assembling a portable medical imaging system, comprising providing a gantry ring having a bore; and coupling an image collection apparatus to the gantry, the image collection apparatus comprising at least two x-ray sources and an x-ray detector array, wherein each x-ray source is mounted on the gantry ring at an angle from a normal line at a point on the gantry ring where the source is mounted, and wherein the angle for each source is different.

38. The method of claim 37, wherein the image collection apparatus is rotatably coupled to the gantry ring, and wherein the gantry ring is less than about 20 inches in diameter and less than about 10 inches in width.

39. The method of claim 38, further comprising providing:
(a) a processor comprising computer-executable instructions for:
(i) controlling acquisition of a projection of an object produced by the image collection apparatus;
(ii) controlling movement of the image collection apparatus; and
(iii) generating an image by integrating multiple projections of the object; and
(b) a power source.
